(19) United States
(12) Patent Application Publication
(10) Pub. No.: US 2005/0165288 A1
(43) Pub. Date: Jul. 28, 2005

(54) Systems and methods for treating breast tissue

(75) Inventors: Robert F. Rioux, Ashland, MA (US); Paul DiCarlo, Middleboro, MA (US)

Correspondence Address: Bingham McCuthen, LLP, Three Embarcadero, Suite 1800, San Francisco, CA, 94111-4067, US

(73) Assignee: Scimed Life Systems, Inc., Maple Grove, MN (US)

(21) Appl. No.: 10/766,608

(22) Filed: Jan. 27, 2004

(51) Int. Cl. A61N 5/00; A61M 36/00; B64D 47/00; A61B 8/12

(52) U.S. Cl. 600/342; 600/7; 600/437; 600/19

(57) ABSTRACT

A system for treating breast tissue includes a cannula having a proximal end, a distal end, and a lumen extending between the proximal and distal ends, the distal end configured for insertion into a breast duct such that the lumen is in fluid communication with the breast duct, and a tissue diagnostic device disposed within the lumen. A system for treating breast tissue includes a cannula having a proximal end, a distal end, and a lumen extending between the proximal and distal ends, the distal end configured for insertion into a breast duct such that the lumen is in fluid communication with the breast duct, an imaging device for providing imaging functionality to the cannula, and an energy delivery device secured to, or slidably disposed within the lumen of, the cannula.

### FIELD OF THE INVENTION

The present invention pertains to systems and methods for diagnosing and treating cancerous breast tissue.

### BACKGROUND

Each year, many women are diagnosed with breast cancer. It is believed that breast ducts may be the origin of breast cancer cells. When cancer cells are confined to breast ducts, the cancer is classified as intraductal cancer, which is frequently curable. When cancer cells break out of a breast duct, however, the cancer cells become an infiltrating ductal carcinoma, and may gain access to lymphatic spaces and blood vessels in the breast. Such carcinomas are much more difficult to treat and cure. Further, after an initial treatment, the cancer cells may recur. As such, early diagnosis and treatment of intraductal cancer is highly desirable.

To detect the presence of cancerous or pre-cancerous cells, a tiny catheter has been used to extract duct-lining cells from breast ducts in a technique know as ductal lavage. The catheter is inserted into a breast duct at a nipple, and salt water or saline is introduced into the breast duct through the catheter. The breast is then massaged to retrieve the delivered fluid from within the breast duct. If the retrieved fluid carries duct-lining cells, the duct-lining cells can be examined by a cytopathologist to determine if the duct-lining cells are abnormal, e.g., malignant or pre-cancerous.

While the ductal lavage technique allows for extraction of duct-lining cells from breast ducts, it does not allow a physician or technician to analyze the retrieved cells during the same diagnostic session with the patient. Often, it can take days before the patient can find out the cytopathological result, and if it is determined that the retrieved cells are abnormal, the patient often must return to her physician for additional testing and/or treatment. In addition, massaging a patient's breast may not be the most effective way to retrieve the delivered fluid, and the patient may experience discomfort. Further, current treatment of breast cancer, which may include one or both of radiation therapy and open breast surgery (including mastectomy) to remove cancerous breast cells, may not be the most effective or desirable method for removing or destroying cancerous or pre-cancerous breast cells. Application of radiation using an external radiation source may subject healthy breast tissue adjacent to target tissue to unnecessary and harmful radiation. Open breast surgery may subject the patient to risk of complications and even death.

### SUMMARY OF THE INVENTION

In accordance with one embodiment of the invention, a system for treating breast tissue includes a cannula and a tissue diagnostic device. The cannula has a proximal end, a distal end, and a lumen extending between the proximal and distal ends, the distal end configured for insertion into a breast duct such that the lumen is in fluid communication with the breast duct. The tissue diagnostic device is disposed within the lumen. By way of non-limiting example, the tissue diagnostic device can comprise a spectrometer.

In accordance with another embodiment of the invention, a system for treating breast tissue includes a cannula having a proximal end, a distal end, and a lumen extending between the proximal and distal ends, the distal end configured for insertion into a breast duct such that the lumen is in fluid communication with the breast duct. The system also includes an imaging device for providing imaging functionality to the cannula, and an energy delivery device secured to, or slidably disposed within the lumen of, the cannula.

In accordance with another embodiment of the invention, a system for treating breast tissue includes a cannula having a proximal end, a distal end, and a lumen extending between the proximal and distal ends, the distal end configured for insertion into a breast duct such that the lumen is in fluid communication with the breast duct. The system also includes an imaging device for providing imaging functionality to the cannula, a media delivery device coupled to the proximal end of the cannula, and an aspirator coupled to the distal end of the cannula, the aspirator configured to create a suction within the lumen.

In accordance with another embodiment of the invention, a system for treating breast tissue includes a cannula having a proximal end, a distal end, and a first lumen extending between the proximal and distal ends, the distal end configured for insertion into a breast duct such that the first lumen is in fluid communication with the breast duct. The system also includes an energy delivery device located at the distal end of the cannula, a media delivery device coupled to the proximal end of the cannula, and an aspirator coupled to the distal end of the cannula, the aspirator configured to create a suction within the first lumen.

In accordance with one embodiment of the invention, a method of treating breast duct tissue includes inserting a distal end of a cannula into a breast duct, the cannula having an electrode at the distal end, placing a tissue diagnostic device through the cannula into the breast duct, analyzing tissue in the breast duct using the tissue diagnostic device, and ablating tissue in the breast duct using the electrode.

In accordance with another embodiment of the invention, a method of treating breast duct tissue includes inserting a distal end of a cannula into a breast duct, the cannula having a tissue diagnostic device and an energy delivery device, examining tissue or cells at the breast duct using the tissue diagnostic device to determine whether to treat the breast duct, and treating at least a portion of the breast duct using the energy delivery device.

In accordance with another embodiment of the invention, a method of treating breast duct tissue includes delivering an electrically conductive media within a breast duct, and delivering radio frequency energy to an electrode that is in contact with the delivered electrically conductive media.

In accordance with another embodiment of the invention, a method of treating breast duct tissue includes inserting a cannula having imaging functionality into a breast duct, obtaining an image of at least a portion of the breast duct, and treating breast duct tissue identified in the obtained image.

Other and further aspects and features of the invention will be evident from reading the following detailed description of the drawings, which is intended to illustrate, not limit, the invention.

### BRIEF DESCRIPTION OF THE DRAWINGS

Embodiments of the present invention are illustrated by way of example, and not by way of limitation, in the figures of the accompanying drawings, in which like reference numerals refer to like components, and in which:

FIG. 1 illustrates a first system for examining breast tissue;

FIG. 2 illustrates a second system for examining breast tissue, particularly showing the system having an aspirator;

FIG. 3A illustrates a cannula having an ablation electrode;

FIG. 3B illustrates another cannula having an ablation electrode and a return electrode;

FIG. 3C illustrates a further system for treating breast tissue, particularly showing a return electrode being carried by a shaft disposed within a cannula;

FIG. 4A illustrates yet another system for treating breast tissue, particularly showing an ablation device carrying an ablation electrode;

FIG. 4B illustrates still another system for treating breast tissue, particularly showing an ablation device carrying an ablation electrode and a return electrode;

FIG. 5 illustrates a cannula having imaging capability; and

FIG. 6 illustrates a cannula having three lumens that function as working channels.

### DESCRIPTION OF THE ILLUSTRATED EMBODIMENTS

Various embodiments of the present invention are described hereinafter with reference to the figures. It should be noted that the figures are not drawn to scale and that elements of similar structures or functions are represented by like reference numerals throughout the figures. It should also be noted that the figures are only intended to facilitate the description of specific embodiments of the invention. They are not an exhaustive description of the invention or as a limitation on the scope of the invention. In addition, each illustrated embodiment may not incorporate all the aspects or features of the invention, and an aspect or an advantage described in conjunction with a particular embodiment of the present invention is not necessarily limited to that embodiment, but can be included in any of a number of other embodiments, even if not so illustrated.

Cells Extraction and/or Diagnosing

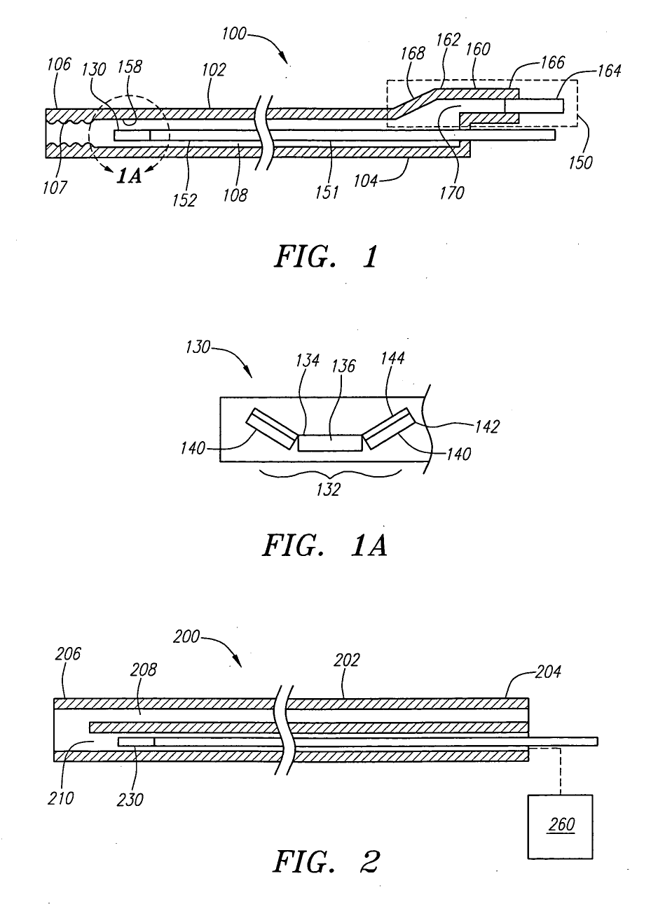

FIG. 1 shows a system 100 for extracting and diagnosing cells from a breast duct. The system 100 includes a cannula 102, a tissue diagnostic device 130, and a media delivery device 150. The cannula 102 is configured to gain access to a lumen of a breast duct, and includes a proximal end 104, a distal end 106, and a lumen 108 extending between the proximal and distal ends 104, 106. The distal end 106 of the cannula 102 has a cross sectional dimension such that the distal end 106 can be inserted into a breast duct. In the illustrated embodiment, the distal end 106 has a cross sectional dimension that is between 0.5 mm and 2.0 mm, and more preferably between 0.7 mm and 1.0 mm. However, the distal end 106 of the cannula 102 may have other dimensions, as long as the distal end 106 can be inserted in a breast duct without causing significant injury to the duct tissue. The distal end 106 of the cannula can also has a tapered profile to allow ease of insertion into a breast duct. In the illustrated embodiment, the distal end 106 of the cannula 102 has a texturized interior surface 107, which can be used to capture cells or tissue from within a breast duct. Alternatively, the interior surface 107 at the distal end 106 of the cannula 102 can be substantially smooth. The cannula 102 can further include a radiopaque marker (not shown) secured to the distal end 106 of the cannula 102, or alternatively, a marker located at an exterior surface of the cannula 102, to assist a placement of the distal end 106 of the cannula 102 during use.

The cannula 102 can be made from a variety of materials, such as, a metal, an alloy, or a polymeric, electrically nonconductive material, like polyethylene, polyurethane, or PEBAX® material (polyurethane and nylon). Alternatively, the cannula 102 can be made from a malleable material, such as stainless steel or aluminum, thereby allowing a physician to change the shape of the cannula 102 before or during an operation. The cannula 102 can also be made from a shape memory alloy, such as Nitinol, or any of the materials disclosed in U.S. Pat. No. 6,485,507. In some embodiments, the distal end 106 is made softer than the proximal portion of the cannula 102 by using different material and/or having a thinner wall thickness. This has the benefit of reducing the risk of injury to tissue that the distal end 106 may come in contact with during an operation. In another embodiment, the cannula 102 can also include a liner secured to an interior wall 158 of the cannula, the liner being composed of a suitable low friction material, e.g., TEFLON™, Polyetheretherketone (PEEK), polyimide, nylon, polyethylene, or other lubricious polymer linings, to reduce surface friction with a device as the device slides within the lumen 108.

The tissue diagnostic device 130 is configured to examine or analyze cells in a breast duct. In one embodiment, the tissue diagnostic device 130 includes a spectrometer 132 for performing an analysis of a tissue's fluorescence characteristics, which can be used in diagnosing conditions such as cancer. The spectrometer 132 includes a source unit 134 for emitting light at a certain frequency or a plurality of frequencies, and a plurality of sensors 140 for measuring light at a plurality of frequencies. The source unit 134 comprises a light source 136, which can be monochromatic or polychromatic. In one embodiment, a tungsten-halogen light is employed as a polychromatic light source. If a polychromatic light source is used, a bandpass filter may be attached. The bandpass filter may allow one or more frequencies to pass through. The frequencies emitted by the source unit 134 are selected to provide data diagnostic of a tissue's condition. In one embodiment, the source unit 134 emits light at a frequency of 435 nm. In other embodiments, the source unit 134 may emit light at a frequency of 420 nm, 490 nm, or any combination thereof. The source unit 134 can be configured to emit light at other frequencies, depending on the characteristics of a tissue desired to be analyzed.

Similarly, the frequencies measured by the sensors 140 are selected to provide data diagnostic of a tissue's condition. In the illustrated embodiment, each of the sensors 140 includes a PIN photodiode 142 that is configured to emit an electrical signal in response to light, and a bandpass filter 144 configured to let through light of certain wavelength(s). In one embodiment, the spectrometer 132 comprises two sensors 140, which measure light at wavelengths of 370 nm and 440 nm, respectively. The sensors 140 can be configured to measure light at other frequencies, depending on the characteristics of a tissue desired to be analyzed.

The tissue diagnostic device 130 and similar devices have been described in U.S. Pat. No. 6,405,073, the entire disclosure of which is expressly incorporated by reference herein. In the illustrated embodiment, the diagnosing device 130 is secured to a shaft 151 at a distal end 152 of the shaft, and is slidably disposed within the lumen 108 of the cannula 102. Alternatively, the diagnosing device 130 can be fixedly secured to the interior wall 158 at the proximal end 104 or the distal end 106 of the cannula 102, in which case, the system 100 does not include the shaft 151. Also, in some embodiments, instead of having the source unit 134 coupled at the distal end 152 of the shaft 151, the source unit 134 is located at a proximal end of the system 100. In such cases, the system 100 includes an illumination channel or scope (e.g., a fiber optic cable) that extends from the source unit 134 to a distal end of the system 100.

In the illustrated embodiment, the media delivery device 150 comprises an tubular extension 160 from the proximal end 104 of the tubular body 102. The tubular extension 160 has a tubular body 162 for storing biocompatible fluid, and a plunger 164. The tubular body 162 includes a proximal end 166, a distal end 168, and a lumen 170 extending between the proximal and distal ends 166, 168. The plunger 164 is at least partially disposed within the lumen 170 at the proximal end 166 of the tubular body 162. In the illustrated embodiment, the distal end 168 of the tubular body 162 is secured to the proximal end 104 of the cannula 102. Alternatively, a tubing (not shown) can be provided to connect the tubular body 162 to the cannula 102. It should be noted that the media delivery device 150 is not limited to the tubular extension 160, and can include any device that is capable of delivering a fluid. For example, in another embodiment, the media delivery device 150 can include a container of the biocompatible fluid and a pump coupled between the cannula 102 and the container. In such case, the pump is activated to convey the fluid from the container into the lumen 108. Furthermore, in some embodiments, the system 100 does not include the media delivery device 150.

When using the system 100 to treat breast tissue, the distal end 106 of the cannula 102 is inserted into the breast duct. After the distal end 106 of the cannula 102 is desirably placed, the plunger 164 is advanced distally to urge the biocompatible fluid from the tubular body 162 into the cannula 102 and the breast duct. After a desired amount of the biocompatible fluid has been delivered, the plunger 164 may be retrieved proximally to pull the delivered biocompatible fluid from the breast duct back into the lumen 108 of the cannula 102. The retrieved fluid may carry duct-lining cells or other cells from the breast duct, which can then be examined by a physician. In addition, or alternatively, if the cannula 102 has the texturized interior surface 107, it can be used to capture tissue or cells. If the system 100 does not include the fluid delivery device 150, then the steps of delivering and retrieving fluid can be performed by other device, or alternatively, be not performed.

Next, the tissue diagnostic device 130 is used to examine or analyze cells in the breast duct to determine if they are cancerous or other types of cells. In the illustrated embodiment, the cells in the breast duct are caused to fluoresce at certain wavelengths, such as of 440 nm and 370 nm, when illuminated by light (e.g. at 300 nm) from the source unit 134. In such case, the bandpass filters 144 let through light of 440 nm and 370 nm, respectively. The PIN photodiodes 142 emit an electrical signal in response to light. The strength of their signals is proportional to the intensity of the light shining on them. The electrical signals can be sent through low pass filters (not shown), which serve to increase the signal-to-noise ration of the output of the PIN photodiodes 142, and then to amplifiers for amplifying the signals. The amplified signals are then transmitted to a processor (not shown), such as a microprocessor, which determines a characteristic of the collected cells. In one embodiment, the processor determines a numeric result, C, which is calculated according to the following formula: C=A * (cells autofluorescence at a first wavelength)+B * (cells autofluorescence at a second wavelength), where A and B are constants set according to the relative autofluorescent characteristics of normal and cancerous cells. If C is above some threshold value, T, then the cells are diagnosed as abnormal or cancerous. The above-described method and similar methods for analyzing cells or tissue have been described in U.S. Pat. No. 6,405,073 referenced herein. As can be seen, the system 100 has the benefit of allowing a physician to retrieve cells from a breast duct and/or analyze cells in the breast duct.

In another embodiment, instead of using the media delivery device 150 to deliver the biocompatible fluid, the biocompatible fluid can be poured into the lumen 108 at the proximal end 104 of the cannula 102. In such case, the system 100 does not include the media delivery device 150. When a desired amount of the biocompatible fluid has been delivered into the breast duct, the breast tissue can be massaged to urge the delivered biocompatible fluid back into the lumen 108 of the cannula 102. The retrieved fluid may carry duct-lining cells or other cells from the breast duct. The steps of delivering fluid into the breast duct and collecting cells can be repeated until a desired amount of cells have been obtained.

In the above-described embodiment, the same lumen 108 is being used to deliver and retrieve the biocompatible fluid. However, this needs not be the case. FIG. 2 shows another system 200 that includes a cannula 202, a tissue diagnostic device 230, and an aspirator 260. The cannula 202 is similar to the cannula 102 discussed previously except that the cannula 202 has a plurality of lumens. In the illustrated embodiment, the cannula 202 has a proximal end 204, a distal end 206, and a first lumen 208 and a second lumen 210 extending between the proximal and distal ends 204, 206. The first lumen 208 can be used to deliver biocompatible fluid to within a breast duct, and the second lumen 210 can be used to retrieve the delivered biocompatible fluid from the breast duct. The aspirator 260 is coupled to the proximal end 204 of the cannula 202 such that when the aspirator 260 is activated, a suction force is created within the second lumen 210 to pull the delivered biocompatible fluid from the breast duct. The tissue diagnostic device 230 is the same as the tissue diagnostic device 130 of FIG. 1. Using the aspirator 260 to retrieve the delivered fluid may reduce or eliminate a discomfort that a patient may otherwise feel when a massaging technique is used to retrieve the fluid. In an alternative embodiment, the system 200 does not include the aspirator 260, in which case, the delivered fluid can be purged from the breast duct by massaging breast tissue, as similarly discussed previously. In some embodiments, instead of delivering a biocompatible fluid, either or both of the lumens 208, 210 can be used to deliver a therapeutic agent, a toxic agent, or a necrosing agent for treatment of the breast duct.

Tissue ablation

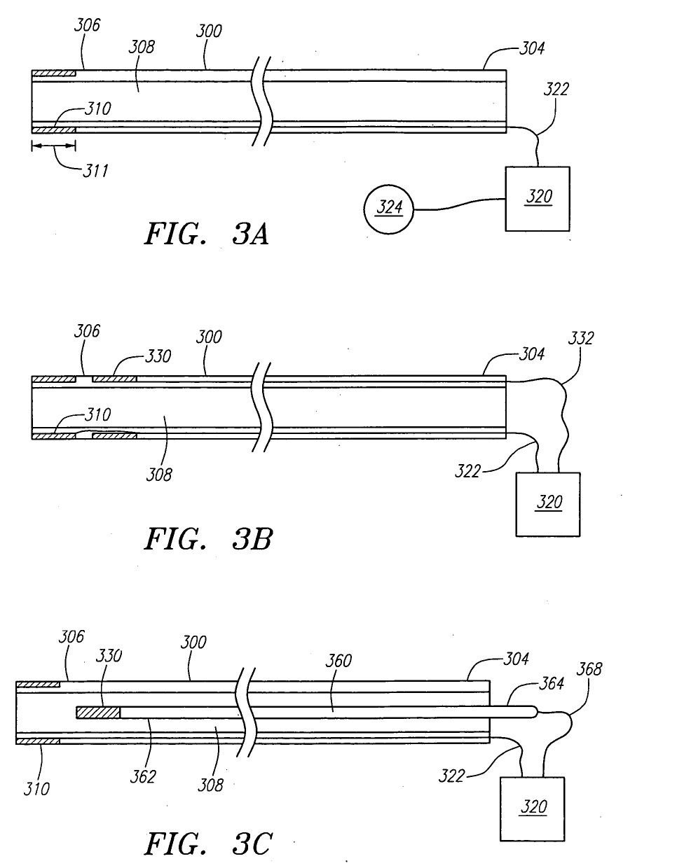

In the above described system 100, the cannula 102 is used to deliver and retrieve biocompatible fluid to and from a breast duct. However, the cannula 102 (or other cannulas described herein) can further include an energy delivery device, such as an ablation device, for delivering energy to tissue. FIG. 3A shows a cannula 300 that can be used with any of the embodiments of the system described herein. The cannula 300 includes a proximal end 304, a distal end 306, and a lumen 308 extending between the proximal and distal ends 304, 306. Although one lumen 308 is shown, in alternative embodiments, the cannula 300 can include a plurality of lumens. The cannula 300 further includes an electrode 310 carried at the distal end 306 of the cannula 300. In the illustrated embodiment, the electrode 310 has a ring shape, and a length 311 that is approximately between 2.0 mm to 3.0 mm. Alternatively, the electrode 310 can have other shapes and/or dimensions. The electrode 310 can be secured to the cannula 300 by a glue or other suitable adhesive, or alternatively, can be an un-insulated portion of the cannula 300.

During use, the distal end 306 of the cannula 300 is inserted into a breast duct. If the tissue diagnostic device 130 is provided, then the cannula 300 can be used to analyze duct tissue, as similarly discussed previously. If it is determined that the breast duct contains abnormal cells, the distal end 306 of the cannula 300 can be further positioned (e.g., advanced distally or retrieved proximally) such that the electrode 310 is positioned adjacent target tissue, such as abnormal or cancerous tissue, to be treated. In the illustrated embodiment, the electrode 310 delivers radio frequency energy to target tissue (e.g., breast duct tissue) in a unipolar arrangement. In such case, the electrode 310 serves as an ablation electrode that is electrically coupled to a generator 320 via a wire 322, while a ground pad 324 is secured to a ground port of the generator. The ground pad 324 is placed on or attached to a patient's skin, such as a breast skin, and completes a current path with the electrode 310. The generator 320 delivers energy to the electrode 310 to ablate the target tissue.

In an alternative embodiment, the electrode 310 can apply energy to a target tissue in a bipolar arrangement. In such case, the cannula 300 includes a second electrode 330 that is located adjacent to the electrode 310 (FIG. 3B). In the illustrated embodiment, the second electrode 330 has a shape of a ring, and is located proximal to the electrode 310. Alternatively, the second electrode 330 can have other shapes and can be secured to the cannula 302 at other locations. The second electrode 330 is electrically secured to the generator 320 via a second wire 332. During use, both electrodes 310, 320 are inserted into a breast duct of a patient, and energy is delivered to one of the electrodes 310, 320 while the other of the electrodes 310, 320 completes the current path. Such configuration can be used to ablate the target tissue at the breast duct.

Instead of being secured to the cannula 302, in another embodiment, the second electrode 330 can be secured to a shaft 360 at a distal end 362 of the shaft 360 (FIG. 3C). In the illustrated embodiment, the second electrode 330 has an elongated shape that is between 2.0 mm to 3.0 mm in length, and a cross sectional dimension that allows the electrode 330 to be inserted into the lumen 308 of the cannula 300. Alternatively, the second electrode 330 can have other shapes or dimensions. A cable 368 is provided that electrically connects the second electrode 330 to the generator 320. In such case, the shaft 360 is slidable within the lumen 308 of the cannula 302 (or another lumen if the cannula 302 has a plurality of lumens), and the second electrode 330 can be positioned by manipulating a proximal end 364 of the shaft 360 during use. For examples, the second electrode 330 can be positioned proximal or distal to the electrode 310, thereby creating a current path that is respectively within or outside the lumen 308 of the cannula 300. In another embodiment, a steering device (not shown) for steering the distal end 362 of the shaft 360 can be provided and secured to the proximal end 364 of the shaft 360. During use, the steering device can be operated to navigate the distal end 362 of the shaft 360 such that the second electrode 330 can be desirably placed. Steering devices have been described in U.S. Pat. Nos. 5,254,088, 5,336,182, 5,358,478, 5,364,351, 5,395,327, 5,456,664, 5,531,686, 6,033,378, and 6,485,455, the entire disclosures of which are expressly incorporated by reference herein.

In some cases, it may be desirable to apply energy to breast tissue that cannot be directly reached by the electrode. In such cases, the lumen 308 of the cannula 302 (or another lumen if the cannula 302 has a plurality of lumens) can be used to deliver electrically conductive fluid, such as a solution that contains ions, into the breast duct to reach target tissue. The delivered conductive fluid can help transmit energy (e.g., ablation energy) from the electrode, and assist delivering of energy to the target tissue that otherwise cannot be reached directly by the electrode.

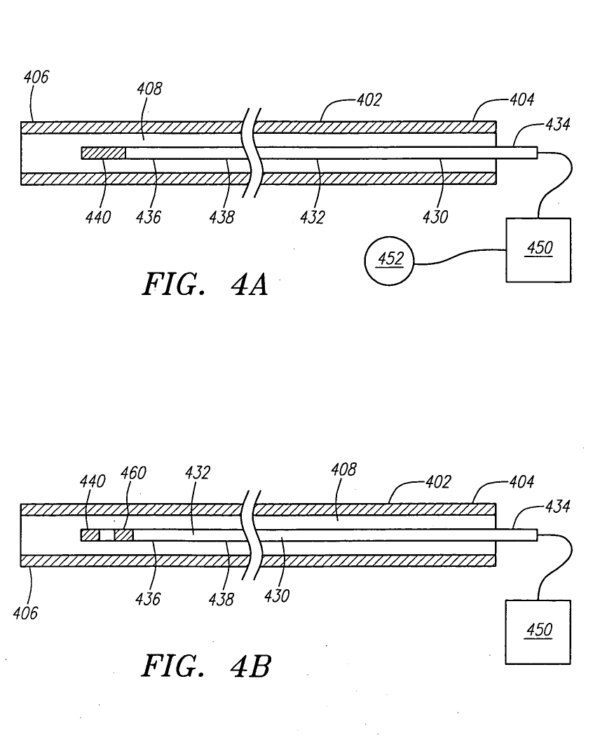

In the above-described embodiments, the cannula 300 includes the electrode 310, and serves as an energy delivery device. In alternative embodiments, the cannula 300 does not include the electrode 310. Instead, a separate energy delivery device can be provided. FIG. 4A shows a system 400 that includes a cannula 402 and an energy delivery system 420. The cannula 402 includes a proximal end 404, a distal end 406, and a lumen 408 extending between the proximal and distal ends 404, 406. The cannula 402 is similar to the cannula 102 discussed previously. In an alternative embodiment, the cannula 402 can include additional lumen(s). The energy delivery system 420 includes an energy delivery device 430, a ground pad 452, and a RF generator. The energy delivery device 430 includes a shaft 432 having a proximal end 434, a distal end 436, and a body 438 extending between the proximal and distal ends 434, 436. The energy delivery device 430 also includes an electrode 440 secured to the distal end 436 of the shaft 432. In the illustrated embodiment, the electrode 440 has a relatively short profile along the length of the cannula 402. Alternatively, the electrode 440 can be an ablation rod made from an elastic or mallable material such that the electrode 440 can conform to a profile of a breast duct while the electrode 440 is being inserted into a breast duct. The energy delivery device 430 can further include a steering device secured to the proximal end 434 of the shaft 432, as similarly discussed previously.

During use, the distal end 406 of the cannula 420 is inserted into a breast duct. The energy delivery device 430 is electrically coupled to the generator 450 and the ground pad 452 is placed at or attached to a patient's skin. The energy delivery device 430 is next inserted into the lumen 408 of the cannula 402, and the energy delivery device 430 is distally advanced until it exits from the distal end 406 of the cannula 420. The distal end 436 of the shaft 432 can be further distally advanced by manipulating the proximal end 434 of the shaft 432 until the electrode 440 is adjacent a target tissue. If a steering device is provided, the steering device can be operated to navigate the distal end 436 of the shaft 432. When the electrode 440 is desirably placed, energy is then delivered to the electrode 440 to ablate the target tissue in a unipolar arrangement.

In an alternative embodiment, the energy delivery device 430 can include a second electrode 460 that cooperates with the electrode 440 to ablate a target tissue in a bipolar arrangement, as similar discussed previously (FIG. 4B). During use, both electrodes 440, 460 are electrically coupled to the generator 450, and are inserted into a breast duct via the cannula 402. Energy is then delivered by the electrodes 440, 460 in a bipolar arrangement to ablate a target tissue. Devices and methods for ablating or treating tissue in unipolar and bipolar arrangements are well known in the art, and therefore, would not be discussed in further detail herein.

It should be noted that besides delivering radio-frequency energy to treat tissue, in alternative embodiments, any of the systems described herein can deliver energy to treat tissue using other techniques. For examples, in an alternative embodiment, instead of an electrode, the energy delivery device can include an optical fiber configured to deliver laser energy to target tissue. The optical fiber can be inserted into a lumen (e.g., lumen 108) of a cannula (e.g., cannula 102) to access a breast duct. The optical fiber can then deliver a narrow beam to selectively ablate a tissue within the breast duct. Alternatively, the optical fiber can be secured to or partially embedded within a wall of the cannula 102. In another embodiment, the ablation device can include an ultrasonic transducer that can be used to deliver ultrasonic energy to target tissue. The ultrasonic transducer can be slidably disposed within the lumen 108 of the cannula 102, or alternatively, be secured to or partially embedded within a wall of the cannula 102. In another embodiment, a device having a thermal tip that is electrically heated through resistance can be provided to apply energy to target tissue.

Besides tissue ablation, any of the systems described herein can destroy or remove abnormal breast tissue using other techniques. In another embodiment, the cannula 102 can be used to deliver one or more radioactive seed into a breast duct. The radioactive seed releases radiation over time and kills target breast tissue. In yet another embodiment, the cannula 102 can be used to deliver an agent, such as a medicine, a toxic agent, a therapeutic agent, a necrosing agent, or a heated fluid, to a breast duct to thereby destroy target tissue. In such case, the cannula 102 can further include a bio-absorbable plug disposed within the lumen 108 of the cannula 102 to control a release of medication or chemicals.

Tissue Imaging

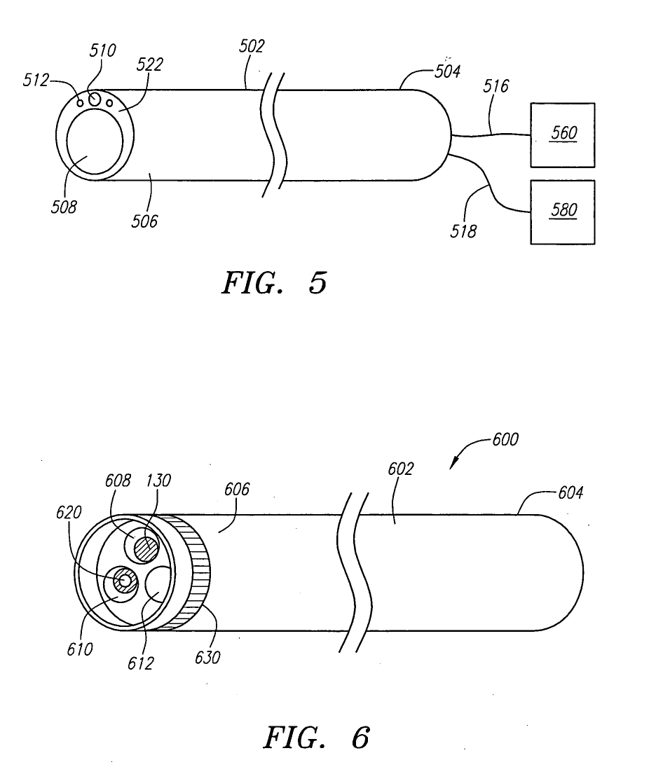

Any of the embodiments of the system described herein can further include an imaging device and/or a light source. FIG. 5 shows a cannula 502 that may be used instead of the cannulas described previously. The cannula 502 has a proximal end 504, a distal end 506, and a lumen 508 extending between the proximal and distal ends 504, 506. The cannula 502 is coupled to an imaging device 560 (e.g., a charge coupled device (CCD) camera) that provides imaging functionality to the cannula 502, and a light source 580 that provides optical viewing functionality to the cannula 502. The cannula 502 is configured to be partially inserted into a breast duct in order to provide access to, and imaging of, a target area within a breast.

The cannula 502 also includes an imaging window 510 located at the distal end 506 of the cannula 502, and an imaging cable 516 housed within a wall 522 of the cannula 502. The imaging cable 516 couples the imaging device 560 to the imaging window 510, so that the cannula 502 is capable of sensing images in the vicinity of the distal end 506 of the cannula 502. The cannula 502 further includes one or more optical windows 512 (in this case, two) located at the distal end 506 of the cannula 502, and fiber-optic cables 518 housed within the wall 522 of the cannula 502. The fiber-optic cables 518 couple the light source 580 to the optical windows 512, so that the cannula 502 is capable of supplying light to illuminate objects that are being imaged. Alternatively, instead of being housed within the wall 522, the fiber-optic cables 518 can be slidably disposed within the lumen 508 of the cannula 502.

It should be noted that the types of imaging device should not be limited to the example discussed previously, and that other types of imaging device can also be used instead. For example, in an alternative embodiment, a ductoscope or an endoscope can be used to provide imaging functionality to the cannula 502. The endoscope can be slidably disposed within the lumen 508 of the cannula 502, or alternatively, be housed within the wall 522 of the cannula 502. Furthermore, in an alternative embodiment, the imaging device itself can include a light source. In such case, the cannula 502 does not include the optical windows 512.

Although various features of the present invention have been discussed with reference to different embodiments, it is understood by those skilled in the art that a feature of an embodiment can be combined with another feature of another embodiment of the system described herein. For example, FIG. 6 shows a system 600 that includes a cannula 602 having a proximal end 604, a distal end 606, and a first lumen 608, a second lumen 610, and a third lumen 612 extending between the proximal and distal ends 604, 606. Each of the lumens 608, 610, and 612 functions as a working channel that can be used to deliver a fluid or a device. In one embodiment, the first lumen 608 can be used to house the tissue diagnostic device 130, and the second lumen 610 can be used to house an imaging device 620, such as a ductoscope or an endoscope. The third lumen 612 can be used to deliver fluid, such as a biocompatible fluid, a toxic agent, a therapeutic agent, or a necrosing agent, to the breast duct. Alternatively, the third lumen 612 can be used to house an additional device, such as a guidewire or an ablation device. In some embodiments, the system 600 only includes the lumens 608, 610, and does not include the third lumen 612. In alternative embodiments, the cannula 602 can include other numbers of lumen, and the lumens can be used for different combination of purposes described herein or for other purposes not described herein. In the illustrated embodiments, the system 600 also includes an electrode 630 for ablation of a duct tissue. Alternatively, the system 600 does not include the electrode 630, in which case, any of the lumens 608, 610, 612 can be used to deliver an agent for treatment of duct tissue.

Although various embodiments of system have been described with reference to treating breast tissue, it should be noted that the same or similar systems can be used to treat tissue at other locations. For examples, any of the above-described systems can be used in a urinary tract, a bile duct, a tear duct, a fallopian tube, a bronchial tube, a colon, or other locations of a patient.

Thus, although different embodiments have been shown and described, it would be apparent to those skilled in the art that many changes and modifications may be made thereunto without the departing from the scope of the invention, which is defined by the following claims and their equivalents.

1. A system for treating breast tissue, comprising:
a cannula having a proximal end, a distal end, and a first lumen extending between the proximal and distal ends, the distal end configured for insertion into a breast duct such that the first lumen is in fluid communication with the breast duct; and
a tissue diagnostic device disposed within the first lumen.

2. The system of claim 1, wherein the tissue diagnostic device comprises a spectrometer.

3. The system of claim 1, further comprising an electrode secured to the distal end of the cannula.

4. The system of claim 1, further comprising an electrode that is slidably disposed within the first lumen.

5. The system of claim 1, further comprising an optical fiber for delivering laser energy, the optical fiber secured to, or slidably disposed within the first lumen of, the cannula.

6. The system of claim 1, further comprising an ultrasonic transducer secured to, or slidably disposed within the first lumen of, the cannula.

7. The system of claim 1, further comprising a media delivery device coupled to the proximal end of the cannula.

8. The system of claim 1, further comprising an aspirator coupled to the proximal end of the cannula.

9. The system of claim 1, the cannula having a second lumen extending between the distal and proximal ends, and further comprising an imaging device secured to, or slidably disposed within, the second lumen.

10. The system of claim 9, wherein the imaging device comprises a CCD camera secured to the cannula.

11. The system of claim 9, wherein the imaging device comprises an endoscope.

12. The system of claim 9, further comprising an electrode secured to the distal end of the cannula.

13. The system of claim 1, the cannula having a second lumen extending between the distal and proximal ends, and further comprising a device slidably disposed within the second lumen, the device selected from the group consisting of an electrode, an optical fiber, and an ultrasonic transducer.

14. The system of claim 1, wherein the cannula is adapted to deliver a substance to the breast duct, the substance selected from the group consisting of a radiation seed, a toxic agent, a therapeutic agent, a necrosing agent, saline, and electrically conductive fluid.

15. A system for treating breast tissue, comprising:
a cannula having a proximal end, a distal end, and a lumen extending between the proximal and distal ends, the distal end configured for insertion into a breast duct such that the lumen is in fluid communication with the breast duct;
an imaging device for providing imaging functionality to the cannula; and
an energy delivery device secured to, or slidably disposed within the lumen of, the cannula.

16. The system of claim 15, wherein the energy delivery device comprises an electrode.

17. The system of claim 15, wherein the energy delivery device comprises an optical fiber for delivering laser energy.

18. The system of claim 15, wherein the energy delivery device comprises an ultrasonic transducer.

19. The system of claim 15, further comprising a media delivery device coupled to the proximal end of the cannula.

20. The system of claim 21, the cannula comprising a plurality of lumens extending between the proximal and distal ends, and further comprising an aspirator coupled to the distal end of the cannula and configured to create a suction within one of the lumens.

21. The system of claim 15, wherein the cannula is adapted to deliver a substance into the breast duct, the substance selected from the group consisting of a radiation seed, a toxic agent, a therapeutic agent, a necrosing agent, saline, and electrically conductive fluid.

22. A system for treating breast tissue, comprising:
a cannula having a proximal end, a distal end, and a lumen extending between the proximal and distal ends, the distal end configured for insertion into a breast duct such that the lumen is in fluid communication with the breast duct;
an imaging device for providing imaging functionality to the cannula;
a media delivery device coupled to the proximal end of the cannula; and
an aspirator coupled to the distal end of the cannula, the aspirator configured to create a suction within the lumen.

23. The system of claim 22, wherein the imaging device is secured to, or slidably disposed in the lumen of, the cannula.

24. The system of claim 23, wherein the imaging device comprises a CCD camera secured to the cannula.

25. The system of claim 22, wherein the imaging device comprises an endoscope.

26. The system of claim 22, wherein the cannula is adapted to deliver a substance into the breast duct, the substance selected from the group consisting of a radiation seed, a toxic agent, a therapeutic agent, a necrosing agent, saline, and electrically conductive fluid.

27. A system for treating breast tissue, comprising:
a cannula having a proximal end, a distal end, and a first lumen extending between the proximal and distal ends, the distal end configured for insertion into a breast duct such that the first lumen is in fluid communication with the breast duct;
an energy delivery device located at the distal end of the cannula;
a media delivery device coupled to the proximal end of the cannula; and
an aspirator coupled to the distal end of the cannula, the aspirator configured to create a suction within the first lumen.

28. The system of claim 27, wherein the energy delivery device is secured to the cannula, or slidably disposed within the first lumen.

29. The system of claim 27, the cannula having a second lumen extending between the proximal and distal ends, wherein the energy delivery device is slidably disposed within the second lumen.

30. The system of claim 27, wherein the energy delivery device comprises an electrode.

31-61. (canceled)
